(19) United States
(12) Patent Application Publication
(10) Pub. No.: US 2002/0192178 A1
(43) Pub. Date: Dec. 19, 2002

(54) Leguminous extract heavy-metal-scavenging anti-pollution agent

(75) Inventors: Pascale Pelletier, Antony (FR); Philippe Catroux, Nogent Sur Marne (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/067,376

(22) Filed: Feb. 7, 2002

(30) Foreign Application Priority Data
Feb. 7, 2001 (FR) 01 01642

(51) Int. Cl. A61K 35/78; A61K 7/42; A61K 7/06; A61K 7/11

(52) U.S. Cl. 424/70.14; 424/59; 424/750

(57) ABSTRACT

The present application relates to the use in topical application of at least one gramineous extract as an anti-pollution agent, and especially as an anti-pollution cosmetic agent. The application also relates to a cosmetic treatment process for protecting the body against the effects of pollution, in particular against the effects of heavy metals, by applying to keratin materials a composition containing, in a physiologically acceptable medium, an effective amount of at least one gramineous extract.

### BACKGROUND OF THE INVENTION

1. FIELD OF THE INVENTION

The present application relates to the use, for example in topical application, of at least one gramineous plant extract as an anti-pollution agent, and to a cosmetic treatment process for protecting the keratin materials against the effects of pollution, in particular against the effects of heavy metals, which comprises the application to keratin materials of a composition containing, in a physiologically acceptable medium, at least one aqueous, alcoholic or aqueous-alcoholic gramineous extract.

2. DISCUSSION OF THE BACKGROUND

Urban environments are regularly subjected to peaks of pollution. An individual in his daily environment, and particularly in an urban zone, may be subjected to a whole range of factors attacking keratin materials, and in particular the skin, the scalp and the hair, by various airborne pollutants. Atmospheric pollutants which are represented largely by the primary and secondary products of combustion represent a major source of environmental oxidative stress. Urban pollution is composed of various types of chemical and xenobiotic products and particles. Three major categories of pollutants which may exert harmful effects on the skin and the hair are as follows: gases, heavy metals and particulate elements which are combustion residues onto which are absorbed a very large number of organic compounds.

It is the outermost tissues that are initially and directly exposed to environmental toxins. The skin is directly and frequently exposed to the pro-oxidative environment. It is particularly sensitive to the action of oxidative stress and its outermost layer serves as a barrier to oxidative damage which may take place. In the majority of circumstances, the oxidizing agent is generally neutralized after reaction with the keratin materials, but the reaction products formed may be responsible for attacks on cells and tissues.

The stratum corneum, the skin's barrier, is the site of contact between the air and skin tissue. The presence of a lipid/protein two-phase structure is a crucial factor of this barrier function of the skin. These elements may react with the oxidizing agents and become impaired, which will promote the desquamation phenomena.

It is known that heavy metals are atmospheric pollutants whose emissions have increased considerably, especially in urban and industrial environments. In addition to certain toxic effects which they cause, heavy metals have the property of reducing the activity of the cellular defense means against free radicals (J. Toxicol. Cut. & Ocular Toxicol., 1987, 6(3), 183-191). Thus, heavy metals aggravate the toxic effects of gaseous pollutants by reducing the efficacy of the natural defense means, and bring about an acceleration of the phenomenon of cell ageing. This is particularly true for keratin materials and especially the skin, the scalp and the hair, which are in direct and permanent contact with the external environment.

Among these urban airborne pollutants of the metallic type that are encountered are metals or metalloid elements such as lead, cadmium, mercury, arsenic (or arsenic-based compounds), chromium (or chromium-based compounds), compounds containing nickel, radon, beryllium, cadmium, silica, lead, etc.), (Environmental Health Perspectives, 1994, 102: 193-210). These elements are generally absorbed onto carbon particles in suspension in the air (especially combustion residues of heat engines).

It has been shown that certain metals can penetrate into the skin and become accumulated (Critical Reviews in Toxicology, 1995, 25: 397-462). The majority of the effects are described in tissues such as the lungs, the kidneys, the brain, etc. At high concentrations, they can induce:

oxidation mechanisms, in particular of the membrane lipids,

a direct cytotoxicity, capable of resulting in cell necrosis,

an alkylation of cell nucleophiles, these mechanisms possibly being the cause of sensitization phenomena or carcinogenesis, (Free Radical Biology and Medicine, 1995, 18, 321-336.; The Journal of Biological Chemistry, 1998, 21, 12703-12709.; Biochemical and Molecular Medicine, 1995, 54, 33-37).

Thus, the harmful effects of pollution on keratin materials affect cell respiration of these keratin materials and are reflected by accelerated ageing of the skin, with a dull complexion and the early formation of wrinkles or fine lines, and also by a reduction in the vigor of the hair, which thus acquires a dull appearance. In addition, due to pollution, the skin and hair become dirty more quickly. Furthermore, pollution can cause irritations and allergic phenomena and inflammation on the skin.

Various antipollution agents have been described to combat these effects of heavy metals. Thus, document EP-A-557 042 describes the use of metallothionines to protect tissues against heavy metals.

### OBJECT OF THE INVENTION

With pollution on the increase, there is a need to find other agents for effectively combating the harmful effect of pollutants on keratin materials and to prevent the adhesion of these pollutants on keratin materials, and in particular to avoid the degradation of cell respiration, the desquamation and accelerated ageing of keratin materials and especially the skin, and also to combat the dull complexion and the early formation of wrinkles and fine lines on the skin, to prevent hair from having a dull appearance and from becoming dirty, and to avoid irritation of the skin and also skin allergy phenomena and skin inflammation. The Inventors have now found, entirely surprisingly, that the use of an aqueous, alcoholic or aqueous-alcoholic gramineous extract makes it possible to protect keratin materials against the effects of pollutants and obtain these objects.

### SUMMARY OF THE INVENTION

Thus, one subject of the invention is the use (general and cosmetic) of at least one aqueous, alcoholic or aqueous-alcoholic gramineous extract as an anti-pollution agent, for example in a composition for topical application to keratin materials. More particularly, a subject of the invention is the cosmetic use of at least one aqueous, alcoholic or aqueous-alcoholic gramineous extract as a heavy metal-scavening agent in a composition for topical application to keratin materials.

The present invention also relates to the use of at least one aqueous, alcoholic or aqueous-alcoholic gramineous extract to prepare a topical-application composition for protecting keratin materials against the harmful effects of pollution. More particularly, the present invention relates to the use of at least one gramineous extract to prepare a topical-application composition for protecting keratin materials against the harmful effects of pollution, such as against the effects of heavy metals.

The present invention also relates to compositions of gramineous extracts associated with instructions or other indicia explaining the composition's use and/or usefulness in the protection of keratin materials against the harmful effects of pollution, such as heavy metals.

### DETAILED DESCRIPTION OF THE INVENTION

According to the invention, the expression “gramineous extract” means an extract from a gramineous plant such as, for example, rices, corns, wheats, barleys, ryes and oats. Preferred is a plant extract from cereals such as, for example, wheat, maize, barley or rice.

The extracts used in the context of the present invention may be aqueous extracts, alcoholic extracts in which the solvent is of one or more mono-alcohols (for example ethanol) and/or polyols, or aqueous-alcoholic extracts in which the solvent consists of a mixture of water and one or more mono-alcohols and/or polyols (such as glycol). Preferably, the extract is an aqueous extract.

To carry out the invention, the gramineous plants may advantageously be chosen from cereals such as maize, wheat, barley or rice. Among these cereals, maize will be chosen more particularly. An extract of maize that is particularly suitable for carrying out the present invention is the aqueous extract of maize sold by the company Solabia under the name Phytovityl C. This extract of maize a contains an active material comprising proteins, amino acids (mainly glutamic acid, proline and alanine), myoinositol, mineral salts and sorbic acid, in a medium comprising water and sorbitol.

The composition according to the invention may comprise one or more gramineous extracts.

The expression “heavy-metal scavenger” means an agent which acts by complexing and/or chelating heavy metals so as to protect the skin and keratin materials and to prevent, attenuate and/or eliminate the deleterious effects of these metals.

In the context of the present invention, the expression “keratin material” means the skin, the scalp, the hair, the eyelashes, the eyebrows, the nails and mucous membranes.

The expression “topical application” means herein an external application to keratin materials, these especially being the skin, the scalp, the eyelashes, the eyebrows, the nails and mucous membranes.

The extract used according to the invention is advantageously present in a sufficient amount. The expression “sufficient amount” (or “effective amount”) means herein an amount such that the protection against pollutants is ensured. This amount may range, for example, from 0.01 to 10% by weight and preferably from 0.05% to 5% by weight of anti-pollution compound (gramenious extract) active material (i.e., not including solvents, etc. and including only materials taken out of the extract source) relative to the total weight of the composition, including 0.1, 0.5, 1, 2, 3, 4, 5, 6, 7, 8 and 9% by weight.

The topical-application compositions, and especially cosmetic compositions, according to the invention preferably contain a physiologically acceptable medium, that is to say a medium that is compatible with the skin, the lips, the scalp, the eyelashes, the eyes, the nails and/or the hair. This physiologically acceptable medium may more particularly comprise water and optionally a physiologically acceptable organic solvent chosen, for example, lower alcohols containing from 1 to 8 carbon atoms and in particular 1 to 6 carbon atoms, for instance ethanol, isopropanol, propanol and butanol; polyethylene glycols containing from 6 to 80 ethylene oxides; polyols, for instance propylene glycol, isoprene glycol, butylene glycol, glycerol and sorbitol. It may also be an anhydrous medium, especially an oily medium containing oils and/or fatty substances other than oils.

When the physiologically acceptable medium is an aqueous medium, it preferably has a pH that is compatible with the skin, ranging for example from 3 to 8 and better still from 4 to 7.

When the composition comprises an aqueous or aqueous-alcoholic medium, it is possible to add a fatty (or oily) phase to this medium, so that the compositions of the invention are softer and more nourishing.

Thus, the compositions according to the invention containing the antipollution agent/heavy-metal scavenger as defined above may be in any form conventionally used for topical application, and especially in the form of aqueous, aqueous-alcoholic or oily solutions, oil-in-water (O/W) or water-in-oil (W/O) or multiple (triple: W/O/W or O/W/O) emulsions, aqueous or oily gels, liquid, pasty or solid anhydrous products, or dispersions of a fatty phase in an aqueous phase with the aid of spherules, these spherules possibly being polymer nanoparticles such as nanospheres and nanocapsules, or lipid vesicles of ionic and/or nonionic type. These compositions may be prepared according to the usual methods, within the skill of the ordinary artisan in view of this disclosure.

In addition, the compositions according to the invention may be more or less fluid and may have any appearance including that of a white or colored cream, an ointment, a milk, a lotion, a serum, a paste or a mousse. They may optionally be applied to the skin in the form of an aerosol. They may also be in solid form and, for example, in the form of a stick.

When the composition according to the invention comprises an oily phase, this phase preferably contains at least one oil. It may also contain other fatty substances.

As oils which can be used in the composition of the invention, mention may be made for example of:

hydrocarbon-based oils of animal origin, such as perhydrosqualene;

hydrocarbon-based plant oils such as liquid triglycerides of fatty acids of 4 to 10 carbon atoms, such as heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil or Karite butter oil;

synthetic esters and ethers in particular of fatty acids, such as the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2- octyldodecyl stearte, erucate or isostearyl isotearyl isostearate; hydroxylated esters such as isostearyl lactate, octyl hydroxystearate, octyldodecyl hydroxystearate, diisostearyl malate, triisocetyl citrate, and fatty alkyl heptanoates, octanoates and decanoates; polyol esters such as propylene glycol dioctanoate, neopentyl glycol diheptanoate or diethylene glycol diisononanoate; and pentaerythritol esters such as pentaerythrityl tetraisostearate;

linear or branched hydrocarbons of mineral or synthetic origin, such as volatile or non-volatile liquid paraffins and derivatives thereof, petroleum jelly, polydecenes or hydrogenated polyisobutene such as parleam oil;

fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecan

partially hydrocarbon-based and/or silicone-based fluoro oils such as those described in document JP-A-2 295 912 (incorporated herein by reference);

silicone oils such as volatile or non-volatile polydimethylsiloxanes (PDMSs) containing a linear or cyclic silicone chain, which are liquid or pasty at room temperature, in particular cyclopolydimethylsiloxanes (cyclomethicones) such as cyclohexasiloxane; polydimethylsiloxanes comprising alkyl, alkoxy or phenyl groups, pendant or at the end of a silicone chain, these groups containing from 2 to 24 carbon atoms; phenylsilicones such as phenyl trimethicones, phenyl dimethicones, phenyltrimethyl-siloxydiphenylsiloxanes, diphenyl dimethicones, diphenylmethyldiphenyltrisiloxanes, 2-phenylethyl trimethylsiloxysilicates and polymethylphenylsiloxanes;

mixtures thereof.

In the list of oils mentioned above, the expression “hydrocarbon-based oil” means any oil predominantly comprising carbon and hydrogen atoms, and optionally ester, ether, fluoro, carboxylic acid and/or alcohol groups.

Other fatty substances which may be present in the oily phase are, for example, fatty acids containing from 8 to 30 carbon atoms, for instance stearic acid, lauric acid, palmitic acid and oleic acid; waxes, for example lanolin, beeswax, carnauba wax, candelilla wax, paraffin wax, lignite wax or microcrystalline waxes, ceresin or ozokerite, synthetic waxes, for instance polyethylene waxes) and Fischer-Tropsch waxes; silicone resins such as trifluoromethyl-C1-4-alkyldimethicone and trifluoropropyldimethicone; and silicone elastomers, for instance the products sold under the names “KSG” by the company Shin-Etsu, under the names “Trefil”, “BY29” or “EPSX” by the company Dow Coming or under the names “Gransil” by the company Grant Industries.

These fatty substances may be chosen in a varied manner by a person skilled in the art in order to prepare a composition having the desired properties, for example consistency or texture properties.

According to one particular embodiment of the invention, the composition containing the gramineous extract is a water-in-oil (W/O) or oil-in-water (O/W) emulsion. The proportion of the oily phase of the emulsion may range from 5% to 80% by weight and preferably from 5% to 50% by weight relative to the total weight of the composition. The oils, the emulsifiers and the coemulsifiers used in the composition in emulsion form are chosen from those conventionally used in cosmetics or dermatology. The emulsifier and the coemulsifier are generally present in the composition in a proportion ranging from 0.3% to 30% by weight and preferably from 0.5% to 20% by weight relative to the total weight of the composition. The emulsion may also contain lipid vesicles.

The emulsions may generally contain at least one emulsifier chosen from amphoteric, anionic, cationic or nonionic emulsifiers, used alone or as a mixture. The emulsifiers are chosen in an appropriate manner depending on the emulsion to be obtained (W/O or O/W emulsion) as is within the skill of the ordinary artisan in view of this disclosure.

For the W/O emulsions, mention may be made, for example, as emulsifiers, of dimethicone copolyols such as the mixture of cyclomethicone and of dimethicone copolyol, sold under the name “DC 5225 C” by the company Dow Coming, and alkyldimethicone copolyols such as the laurylmethicone copolyol sold under the name “Dow Coming 5200 Formulation Aid” by the company Dow Coming, and cetyl dimethicone copolyol sold under the name Abil EM 90® by the company Goldschmidt. Surfactants for W/O emulsions which may also be used include a crosslinked elastomeric solid polyorganosiloxane comprising at least one oxyalkylenated group, such as those obtained according to the procedure of Examples 3, 4 and 8 of document U.S. Pat. No.5,412,004 and of the examples of document U.S. Pat. No. 5,811,487, especially the product of Example 3 (synthesis example) of U.S. Pat. No. 5,412,004, and such as the product sold under the reference KSG 21 by the company Shin Etsu.

For the O/W emulsions, mention may be made, for example, as emulsifiers, of nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; and sugar esters such as the methylglucose stearate sold by Amerchol under the name deglutamate SSE 20, sucrose stearate; and mixtures thereof such as the mixture of glyceryl stearate and PEG-40 stearate.

The cosmetic or dermatological composition of the invention may also contain adjuvants that are common in cosmetics or dermatology, such as hydrophilic or lipophilic gelling agents, hydrophilic or lipophilic active agents, preserving agents, antioxidants, solvents, fragrances, fillers, UV screening agents, bactericides, odor absorbers, dyestuffs and salts. The amounts of these various adjuvants are those conventionally used in the field under consideration, and are, for example, from 0.01 to 20% of the total weight of the composition. Depending on their nature, these adjuvants may be introduced into the fatty phase, into the aqueous phase and/or into the lipid spherules.

As fillers which may be used in the composition of the invention, mention may be made, for example, besides pigments, of silica powder; talc; polyamide particles and especially those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; expanded powders such as hollow microspheres, and especially the microspheres sold under the name Expancel by the company Kemanord Plast or under the name Micropearl F 80 ED by the company Matsumoto; silicone resin microbeads such as those sold under the name Tospearl by the company Toshiba Silicone; and mixtures thereof. These fillers may be present in amounts ranging from 0% to 20% by weight and preferably from 1% to 10% by weight relative to the total weight of the composition.

According to one preferred embodiment of the invention, the composition used according to the invention may contain at least one UV screening agent (or sunscreen) which may be a chemical screening agent or a physical sunblock or a mixture of such screening agents. By way of illustration and in a non-limiting manner, mention may be made of the following families (the names correspond to the CTFA nomenclature for screening agents): anthranilates, in particular methyl anthranilate; benzophenones, in particular benzophenone-1, benzophenone-3, benzophenone-5, benzophenone-6, benzophenone-8, benzophenone-9 and benzophenone-12, and preferentially benzophenone-2 (Oxybenzone) or Benzophenone-4 (Uvinul MS40 available from BASF); benzylidenecamphors, in particular 3-benzylidenecamphor, benzylidenecamphorsulphonic acid, camphor benzalkonium methosulphate, polyacrylamidomethyl-benzylidenecamphor, terephthalylidenedicamphorsulphonic acid, and preferentially 4-methylbenzylidenecamphor (Eusolex 6300 available from Merck); benzimidazoles, in particular benzimidazilate (Neo Heliopan AP available from Haarmann & Reimer) or phenylbenzimidazolesulphonic acid (Eusolex 232 available from Merck); benzotriazoles, in particular drometrizole trisiloxane or methylenebis-benzotriazoly-tetramethylbutylphenol (Tinosorb M available from Ciba); cinnamates, in particular cinoxate, DEA methoxycinnamate, diisopropyl methylcinnamate, glyceryl ethylhexanoate dimethoxycinnamate, isopropyl methoxycinnamate and isoamyl cinnamate, and preferentially ethocrylene (Uvinul N35 available from BASF), octyl methoxycinnamate (Parsol MCX available from Hoffmann La Roche), or octocrylene (Uvinul 539 available from BASF); dibenzoylmethanes, in particular butylmethoxydibenzoylmethane (Parsol 1789); imidazolines, in particular ethylhexyldimethoxy-benzylidenedioxoimidazoline; PABAs, in particular ethyl dihydroxypropyl PABA, ethylhexyldimethyl PABA, glyceryl PABA, PABA and PEG-25 PABA, and preferentially diethylhexylbutamidotriazone (Uvasorb HEB available from 3V Sigma), ethylhexyltriazone (Uvinul T150 available from BASF) or ethyl PABA (benzocaine); salicylates, in particular dipropylene glycol salicylate, ethylhexyl salicylate, homosalate, or TEA salicylate; triazines, in particular anisotriazine (Tinosorb S from Ciba); drometrizole trisiloxane, zinc oxide and titanium dioxide.

Examples of UV screening agents that are particularly suitable for use in the present invention are:

the butylmethoxydibenzoylmethane sold in particular by the company Hoffmann-LaRoche under the name Parsol 1789,

the octocrylene sold in particular by the company BASF under the name Uvinul N539,

the octyl salicylate sold in particular by the company Haarmann-Reimer under the name Neo Heliopan OS,

the octyl methoxycinnamate sold in particular by the compound Hoffmann-LaRoche under the name Parsol MCX,

the phenylbenzimidazolesulphonic acid sold in particular by the company Merck under the name Eusolex 232,

oxybenzones such as benzophenones-3, -4 or -5,

benzotriazole silicones and in particular drometrizole trisiloxane,

terephthalylidenedicamphorsulphonic acid, and

titanium oxide or zinc oxide, in the form of microparticles or nanoparticles that are optionally coated.

Benzophenone-3, terephthalylidenedicamphor-sulphonic acid, octyl methoxycinnamate, phenylbenzimidazolesulphonic acid, drometrizole trisiloxane, 4-methylbenzylidenecamphor, anisotriazine, octocrylene, butylmethoxydibenzoylmethane, zinc oxide and/or titanium dioxide is preferably used as screening agent in the composition of the invention.

The amount of screening agents depends on the intended final use. It may range, for example, from 1% to 20% by weight and better still from 2% to 10% by weight relative to the total weight of the composition.

According to another preferred embodiment according to the invention, the composition may also contain an antioxidant active agent (for example vitamin E).

The compositions used according to the invention may especially constitute a care product and/or make-up product for keratin materials, and especially for the skin. They may be used especially to protect the keratin materials against the effects of pollution and more particularly of heavy metals, especially to improve cell respiration and/or to reduce desquamation and/or to prevent keratin materials and especially the skin from becoming dull or dirty. Topical application amounts are not limited, nor is frequency of application. For example 0.5-3 g of the invention composition can be applied with the fingers to the face, neck, hands, etc. In a preferred embodiment the invention methods are used to protect the keratin of those people in need of such heavy metal/pollution protection. Included are people desirous of such specific benefits and those being treated for a condition that would benefit from such protection.

Thus, another subject of the invention consists of a cosmetic treatment process for protecting keratin materials against the effects of pollution and in particular against the effects of heavy metals, which comprises the application to the keratin materials of a composition containing at least one aqueous, alcoholic or aqueous-alcoholic gramineous extract.

A subject of the invention is also a cosmetic treatment process for keratin materials in order to improve their cell respiration and/or to reduce their desquamation and/or to prevent them from becoming dull and/or dirty, which comprises the application to the keratin materials of a composition containing, in a physiologically acceptable medium, at least one aqueous, alcoholic or aqueous-alcoholic gramineous extract.

The above cosmetic treatment processes will be advantageously carried out using an extract of cereals and more particularly an extract of maize.

Also making up a part of the invention is an article of manufacture comprising the invention compositions as described above in combination with or associated with instructions for use as a keratin protectant against heavy metals and/or pollutants, or in combination with or associated with some indicia regarding the composition's ability to provide such protection. Such instructions and indicia include labels, symbols, explicit instructions for use, descriptors of pollution and/or heavy metal protection ability, etc. Such instructions and indicia can appear on the container containing the invention composition, can be included as part of packaging, etc.

French patent application 0101642 filed Feb. 7, 2001, is incorporated herein by reference, as are all documents referred to herein.

The examples which follow serve to illustrate the invention without, however, being limiting in nature. Depending on the case, the names are the chemical names or the CTFA names (International Cosmetic Ingredient Dictionary and Handbook) and the amounts are in percentages by weight, except where otherwise mentioned.

### EXAMPLE 1:

Demonstration of the protective effect of an extract of maize on keratinocytes in culture with respect to a representative heavy metal: cadmium.

### Experimental Protocol

Principle

Heavy metals such as cadmium, nickel, lead, mercury, etc. can cause cytotoxicity on the cells of various organs including the skin. The cytotoxic effect of cadmium on human keratinocytes in culture was evaluated by a technique for measuring the cell viability: the Neutral Red incorporation test (Culture Methods. 1984, 9, 7-9). This method makes it possible to demonstrate a protective effect of the molecule.

Inoculation of the Cells

The study was performed on a monolayer culture of immortalized human keratinocytes (DK7-NR) by the Nestle research center. The cells are inoculated on D-3 in 96-well culture dishes at a rate of 25 000 cells/cm² in 100 μ1 of culture medium (medium defined without calf serum, NR2, Biofluids). The incubations are performed in an incubator at 37° C. in humid atmosphere enriched with 5% CO₂.

Treatment with the pollutant in the presence of the protective agent:

The cells are treated [lacuna] 24 hours with increasing concentrations (0, 10, 25, 50, 75, 100, 150 and 200 μM) of cadmium chloride (CdC1₂) alone, so as to determine its cytotoxicity. In parallel, a treatment is performed under the same conditions, but in the presence of the extract of maize (Phytovityl® at 0.5% and 1%, non-cytotoxic concentrations for the cells). The incubations are performed in an incubator at 37° C., 5% CO₂ in a humid atmosphere.

Measurement of the Cell Viability

At the end of the contact period, the cell viability is determined by means of a Neutral Red (POS 55/006) incorporation test, with spectrophotometric reading at 500 nm. Briefly:

rinse the cells with PBS buffer in order to remove the treatment solutions

add 100 μ1 per well of a 0.5 mg/ml solution of neutral red to the culture medium

incubate at 37° C., 5% CO₂, humid atmosphere for 3 hours,

rinse with PBS

fix with a formol/calcium solution for 1 minute,

extract the neutral red with 100 μl/well of an ethanol/acetic acid solution,

read the optical density on a spectrophotometer at 550 nm,

calculate the concentration of CdC1₂ inducing a 50% decrease in viability=IC₅₀.

Results

A preliminary study of cytotoxicity on DK7-NR keratinocytes was performed (neutral red incorporation test). After contact for 24 hours, the maximum non-cytotoxic concentration of the extract of maize is 1%. Its protective effect was evaluated at concentrations of less than or equal to 1% (W/V).

The results obtained show that the extract of maize exerts a significant protective effect against the toxicity of cadmium. This protective effect is clearly dose-dependent as shown by the table below:

| Concentration of | |
|---|---|
| extract of maize (%) | IC₅₀ (μM) |
| 0 | 37.7 |
| 0.5 | 89.8 |
| 1 | 139.2 |

### COMPOSITION EXAMPLES

### EXAMPLE 2: O/W EMULSION

Phase A (fatty)

| | |
|---|---|
| Phase A (fatty): | |
| Monodiglyceryl stearate | 3 g |
| Liquid petroleum jelly | 3 g |
| Cetyl alcohol | 5 g |
| Phase B (aqueous phase): | |
| Polyethylene glycol oxyethylenated with | 3 g |
| 50 mol of ethylene oxide | |
| Water qs | 100 g |
| Phase C | |
| Extract of maize (Phytovityl ®) | 5 g |
| Water | l0 g |

Procedure

The fatty phase (A) and the aqueous phase (B) are prepared separately and heated to 70° C. The fatty phase is poured into the aqueous phase with stirring. The emulsification is continued for 10 minutes and the mixture is then cooled slowly with stirring to a temperature of 40° C. Phase C is added and the cooling is continued. A cream that can be applied to the skin to protect it against the effects of pollution is obtained.

### EXAMPLE 3

The composition below is formulated in a conventional manner:

| | | |
|---|---|---|
| Extract of maize (Phytovityl ®) | 1 | g |
| Octyl palmitate | 10 | g |
| Glyceryl isostearate | 4 | g |
| Liquid petroleum jelly | 20 | g |
| Sorbitol | 2 | g |
| Vitamin E | 1 | g |
| Glycerol | 3 | g |
| Water qs | 100 | g |

### EXAMPLE 4

The composition below is formulated in a conventional manner:

| | | |
|---|---|---|
| Extract of maize (Phytovityl ®) | 1 | g |
| Octyl palmitate | 10 | g |
| Glyceryl isostearate | 4 | g |
| Purcellin oil | 23 | g |
| Vitamin E | 1 | g |
| Glycerol | 3 | g |
| Water qs | 100 | g |

### EXAMPLE 5 Gel

| | | |
|---|---|---|
| Phase A: | | |
| Water | 10 | g |
| Extract of maize (Phytovityl ®) | 5 | g |
| Phase B: | | |
| Hydroxypropylcellulose | 0.10 | g |
| Carbopol Ultrez 10 | 0.25 | g |
| Polyethylene glycol oxyethylenated with | 3 | g |
| 50 mol of ethylene oxide | | |
| Water qs | 100 | g |
| Phase C | | |
| Triethanolamine qs pH 7 | | |
| Phase D | | |

| | |
|---|---|
| Timiron (titanium-coated mica) | 0.5% |

Procedure

The gelling agents of phase B are dispersed in phase A with vigorous stirring. The mixture obtained is neutralized with phase C. Finally, phase D is dispersed with slow stirring. A gel that can be applied to the skin to protect it against the effects of pollution is obtained.

1. A method for protecting keratin materials against the effects of pollution, comprising applying to a keratin material a composition comprising an effective amount of a gramineous extract.

2. The method according to claim 1, wherein said extract is an extract of a cereal.

3. The method of claim 1, wherein said extract is an aqueous extract.

4. The method according to claim 1, wherein said extract is an extract of maize.

5. The method according to claim 1, wherein said extract is present in an amount of from 0.01 to 10% by weight active material relative to the total weight of said composition.

6. The method according to claim 1, wherein said extract is present in an amount of from 0.05 to 5% by weight active material relative to the total weight of said composition.

7. The method according to claim 1, wherein said extract comprises a mixture of proteins, amino acids, myoinositol, mineral salts and sorbic acid.

8. The method according to claim 1, wherein said composition further comprises at least one antioxidant.

9. The method according to claim 1, wherein said composition further comprises at least one UV screening agent.

10. The method according to claim 1, wherein said pollution comprises heavy metals, and said effective amount is an amount sufficient to provide protection against heavy metals.

11. The method according to claim 1, wherein said keratin material is hair.

12. The method according to claim 1, wherein said effective amount is an amount sufficient to provide protection against heavy metals and other pollutants.

13. The method according to claim 1, wherein said extract is an alcoholic extract.

14. The method according to claim 1, wherein said extract is an aqueous-alcoholic extract.

15. The method according to claim 1, comprising applying said composition to human skin.

16. An article of manufacture comprising:
a composition comprising a gramineous extract; and
instructions for using said composition as a protectant against pollutants.

17. An article of manufacture comprising:
a composition comprising a gramineous extract; and
indicia indicating the ability of said composition to provide protection against pollutants.
